(19) United States
(12) Patent Application Publication
(10) Pub. No.: US 2024/0336482 A1
(43) Pub. Date: Oct. 10, 2024

(54) POWDER COMPOSITION

(71) Applicant: ConvaTec Limited, Flintshire (GB)

(72) Inventors: Hugh Semple Munro, London (GB); Nick Boote, London (GB); Alan Horner, London (GB)

(21) Appl. No.: 18/749,905

(22) Filed: Jun. 21, 2024

Related U.S. Application Data
(63) Continuation of application No. PCT/GB2022/053307, filed on Dec. 20, 2022.

(30) Foreign Application Priority Data
Dec. 22, 2021 (GB) 2118845.3

(51) Int. Cl. C01B 21/50; A61K 33/00; A61K 45/06; A61P 11/00

(52) U.S. Cl. CPC C01B 21/50; A61K 33/00; A61K 45/06; A61P 11/00

(57) ABSTRACT

A solid powder composition comprising one or more particles containing a nitrite salt and a proton source. Also disclosed are pharmaceutical compositions comprising the solid powder composition, methods of producing the solid powder composition, methods of treatment using the solid powder composition, materials and devices comprising the solid powder compositions method of making thereof and methods of implanting thereof into a human or animal body.

The present invention relates to powder compositions including a nitrite salt and a proton source. The present invention also relates to methods of preparing such powder compositions, products including the powder compositions, use of the powder composition in products, and methods of treatment involving the powder compositions.

### BACKGROUND

Nitric oxide (NO) and nitric oxide precursors have been extensively studied as potential pharmaceutical agents. There remain substantial problems in connection with the efficient generation and delivery of nitric oxide, other oxides of nitrogen and precursors thereof to organisms and cells for treatment. A widely adopted system for the generation of nitric oxide relies on the acidification of nitrite salts using a proton source such as an acid to produce initially nitrous acid (HNO₂), which nitrous acid then readily decomposes to nitric oxide and nitrate with hydrogen ions and water. The decomposition can be represented by the following balanced equation (1):

3HNO₂→2NO+NO₃⁻H⁺+H₂O (1)

The acid and nitrite salt are typically provided as separate aqueous solutions at a pre-determined concentrations. These two solutions are thus provided as a two-component system, allowing for the two separate solutions to be combined at the point of need to prevent the release of nitric oxide before required.

The combination of a two-component system at the point of need has the potential to introduce, for example, dosing inaccuracies when the two components are combined. It is desirable to provide a nitrite salt and an acidification source as a single component. In addition, the provision of a single component product may reduce packaging. However, the traditional two-component solution approach in the acidification of nitrite cannot be provided as a single component system because the system would lose a significant proportion of its nitric oxide at the manufacturing stage and could potentially not provide enough nitric oxide at the point of need stage.

### SUMMARY OF THE INVENTION

The present inventors have sought to not only provide a single component system for delivering nitric oxide by acidification of nitrite, but also have sought to provide a solid form of this system that delivers a significant amount of nitric oxide when the solid form is in contact with an aqueous environment.

At its most general, the present invention provides a solid powder composition comprising a nitrite salt and a proton source in solid form and wherein at least part of the nitrite salt and at least part of the proton source are inseparable upon dispersion into a medium or onto a surface. In this way, at least part of the nitrite and at least part of the proton source are held in close proximity (or intimately associated) to provide acidification of the nitrite when in contact with an aqueous environment.

The powder composition may be produced by mixing a solution of the proton source with a solution of the nitrite source and removal of solvent before significant acidification of the nitrite occurs. In this way, the composition may contain one or more particles which contains effective amounts of both the nitrite salt and the proton source in the same particle.

In a first aspect, the present invention provides a solid powder composition comprising one or more particles containing a nitrite salt and a proton source.

In a second aspect, the present invention provides a solid powder composition comprising one or more particles formed by removal of solvent in less than one second (e.g. by spray-drying) and/or under reaction-retarding conditions (e.g. lyophilisation) from a mixture containing a nitrite salt solution and proton source solution.

In a third aspect, the present invention provides a solid powder composition comprising particles coated in a hydrophobic material, wherein the coated particles include a particle containing a nitrite salt and a proton source and the particle is coated with the hydrophobic material.

In this way, the coated particles include nitrite salt and proton source within the same coating.

In a fourth aspect, the present invention provides a pharmaceutical composition including the solid powder composition of the first, second or third aspect and optionally one or more excipients and/or adjuvants.

In a fifth aspect, the present invention provides a method of producing a solid powder composition, the method including the step of removing solvent in less than thirty seconds (e.g. by spray-drying) after mixing of a nitrite solution and a proton source solution to form the solid and/or providing reaction-retarding conditions (e.g. lyophilisation) during solvent removal and before, during and/or immediately after mixing a nitrite salt solution and a proton source solution.

In an sixth aspect, the present invention provides a method of preparing a solid powder composition comprising particles coated in a hydrophobic material, the method includes the step of coating particles containing a nitrite salt and a proton source with a hydrophobic material.

In a seventh aspect, the present invention provides a solid powder composition according to the first, second or third aspects or a pharmaceutical composition according to the fourth aspect for use in a method of treating or preventing a respiratory disease or disorder.

In an eighth aspect, the present invention provides a method of treating or preventing a respiratory disease or disorder, the method includes the administration of a therapeutically effective amount of a solid powder composition according to the first, second or third aspects or a pharmaceutical composition according to the fourth aspect.

In a ninth aspect, the present invention provides a material comprising a substrate and the solid powder composition according to the first, second or third aspects, wherein particles of the solid powder composition are incorporated or encapsulated into the substrate.

In a tenth aspect, the present invention provides a method of incorporating or encapsulating the solid powder composition according to the first or second aspects into a substrate, the method includes the steps of (i) mixing the solid powder composition according to the first, second or third aspects with a non-aqueous or non-polar liquid containing the substrate or substrate precursor to form a liquid-particle mixture and (ii) solidifying the liquid-particle mixture to form a material incorporating or encapsulating the solid powder composition of the first, second third aspects.

In an eleventh aspect, the present invention provides a material or device, wherein the material or device includes a substrate and a spray-dried coating on an exterior surface of the substrate, the spray-dried coating being formed from spray-drying a mixture containing a nitrite salt solution and a proton source solution.

In a twelfth aspect, the present invention provides a material or device, wherein the material or device includes a substrate and a coating on an exterior surface of the substrate, the coating being a homogenous solid containing a nitrite salt and a proton source.

In a thirteenth aspect, the present invention provides a method of providing a material or a device, the method includes the step of spray-drying a mixture containing a nitrite salt solution and a proton source solution onto an exterior surface of a substrate to provide the material or device.

In a fourteenth aspect, the present invention provides a method of implanting a material or device according to the twelfth or thirteenth aspect into a human or animal body.

### DETAILED DESCRIPTION

The present invention will now be described in more detail. The examples and the following figures provide exemplification of the invention.

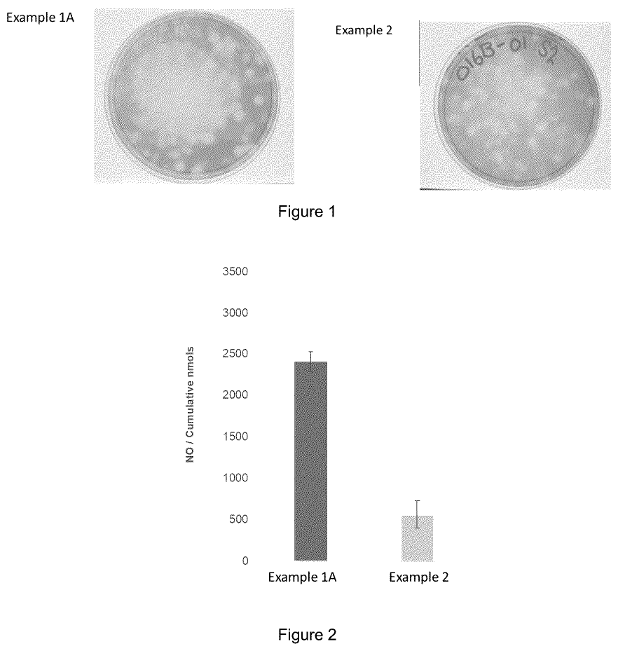

FIG. 1 shows the deposition pattern of powder of Examples 1A and 2 on agarose with Hanks' balanced salt solution and a pH indicator (phenol red).

FIG. 2 shows the cumulative NO generation for Examples 1A and 2.

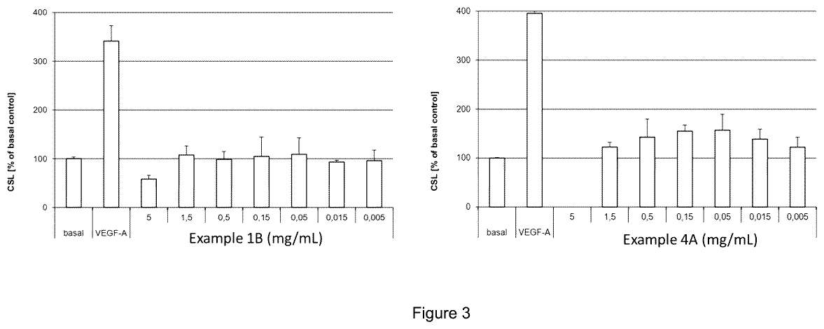

FIG. 3 shows the sprouting intensity of HUVEC spheroids treated with the Examples 1B and 4A quantitated by an image analysis system to determine the cumulative sprout length per spheroid (CSL) relative to the basal control.

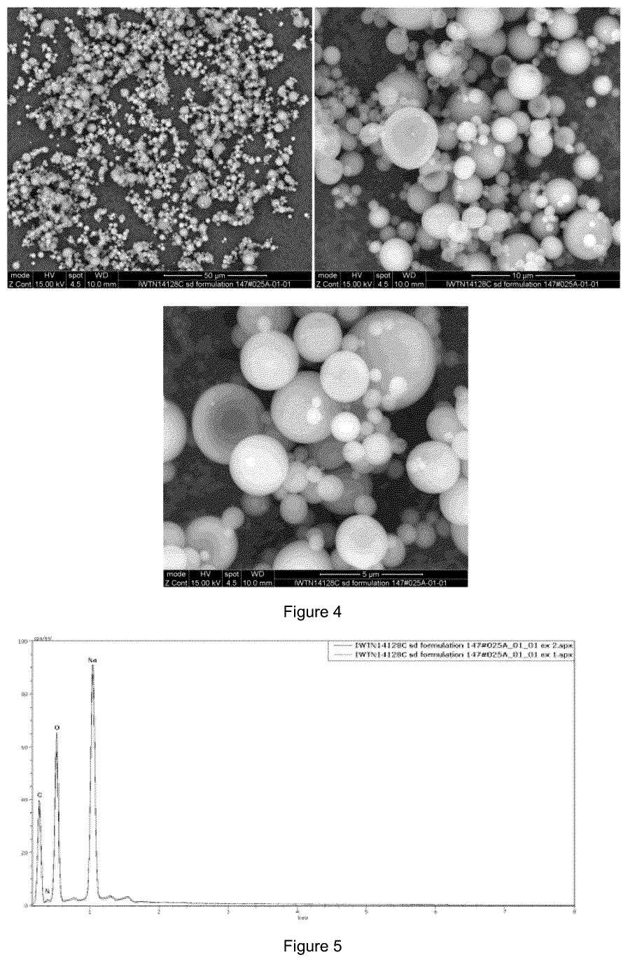

FIG. 4 shows SEM images of spray-dried powders as described herein.

FIG. 5 shows EDX analysis of spray-dried powders as described herein.

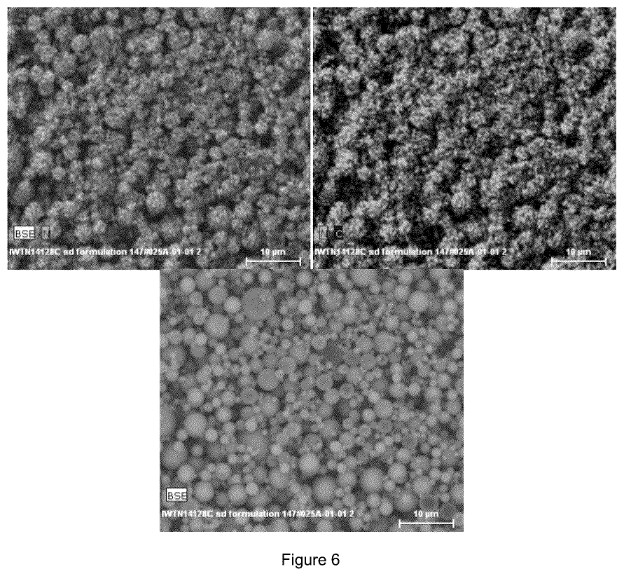

FIG. 6 show EDX maps for Nitrogen (green) against a backscattered electron image (BSE), and Nitrogen against Carbon (blue) at a microscope magnification of ×2000 for spray dried powders as described herein.

The reaction between one or more nitrite salt and a proton source to generate nitric oxide, optionally other oxides of nitrogen and/or optionally precursors thereof is referred to herein as the “NOx generating reaction” or the “reaction to generate NOx” or like wording, and “NOx” is used to refer to the products of the acidification of nitrite, particularly nitric oxide, other oxides of nitrogen and precursors thereof both individually and collectively in any combination. It will be understood that each component of the generated NOx can be evolved as a gas, or can pass into solution in the reaction mixture, or can initially pass into solution and subsequently be evolved as a gas, or any combination thereof.

The term “about” is used herein to denote that the numerical value is not strictly limiting and the skilled person will understand that the value may extend above or below (as appropriate) the exact value in line with the skilled person's understanding of the value. The term “about” may signify a value that is up to ±10% of the value.

Particle size as described herein refers to the volume mean diameter (VMD), unless stated otherwise.

### Solid Powder Compositions

The solid powder compositions of the present invention include a nitrite salt and a proton source. In this way, the solid powder compositions may release nitric oxide by the acidification of the nitrite salt upon exposure to an aqueous environment or to moisture in the atmosphere.

### Nitrite Salt

The choice of nitrite salt is not particularly limited. The nitrite salt may be selected from one or more alkali metal nitrite salts or alkaline metal nitrite salts. For example, the one or more nitrite salt may be selected from LiNO₂, NaNO₂, KNO₂, RbNO₂, CsNO₂, FrNO₂, AgNO₂, Be(NO₂)₂, Mg(NO₂)₂, Ca(NO₂)₂, Sr(NO₂)₂, Mn(NO₂)₂, Ba(NO₂)₂, Ra(NO₂)₂ and any mixture thereof. The nitrite salt may be NaNO₂ or KNO₂. The nitrite salt may be NaNO₂.

The nitrite salt may be a pharmaceutically acceptable grade of nitrite salt. In other words, the nitrite salt may adhere to one or more active pharmacopoeia monographs for the nitrite salt. For example, the nitrite salt may adhere to the monograph of the nitrite salt of one or more of the United States Pharmacopoeia (USP), European Pharmacopoeia or Japanese Pharmacopoeia.

In particular, the nitrite salt used may have one or more of the characteristics as provided in paragraphs [0032] to [0060] to and/or Table 1 in paragraph of WO 2010/093746, the disclosure of which is incorporated herein by reference in its entirety.

### Proton Source

The proton source may be any species capable of acting as a source of protons for the acidification of nitrite. The choice of proton source is not particularly limited. The proton source may be, for example, an acid.

The acid may be selected from one or more organic carboxylic acids or organic non-carboxylic reducing acids.

The expression “organic carboxylic acid” herein refers to any organic acid which contains one or more —COOH group in the molecule. An organic carboxylic acid may be straight-chain or branched. The carboxylic acid may be saturated or unsaturated. The carboxylic acid may be aliphatic or aromatic. The carboxylic acid may be acyclic or cyclic. The carboxylic acid may be a vinylogous carboxylic acid.

The organic carboxylic acid may carry one or more substituents, for example one or more hydroxyl group. Examples of hydroxyl-substituted organic carboxylic acids which may be used in the present disclosure include α-hydroxy-carboxylic acids, β-hydroxy-carboxylic acids and γ-hydroxy-carboxylic acids.

The expression “organic non-carboxylic reducing acid” herein refers to any organic reducing acid which does not contain a —COOH group in the molecule. An organic non-carboxylic reducing acid may be straight-chain or branched. The non-carboxylic reducing acid may be saturated or unsaturated. The non-carboxylic reducing acid may be aliphatic or aromatic. The non-carboxylic reducing acid may be acyclic or cyclic. The non-carboxylic reducing acid may be vinylogous.

The organic non-carboxylic reducing acid may carry one or more substituents, for example one or more hydroxyl group. Examples of hydroxyl-substituted organic non-carboxylic reducing acids which may be used in the present disclosure include the acidic reductones, for example reductic acid (2.3-dihydroxy-2-cyclopentanone).

The one or more organic carboxylic acid or non-carboxylic reducing acid may have a pKa₁ less than about 7.

The one or more organic carboxylic acid may comprise, consist of, or be one or more reducing carboxylic acids. The organic carboxylic acid may, for example, be selected from salicylic acid, acetyl salicylic acid, acetic acid, citric acid, glycolic acid, mandelic acid, tartaric acid, lactic acid, maleic acid, malic acid, benzoic acid, formic acid, propionic acid, α-hydroxypropanoic acid, β-hydroxypropanoic acid, β-hydroxybutyric acid, β-hydroxy-β-butyric acid, naphthoic acid, oleic acid, palmitic acid, pamoic (emboic) acid, stearic acid, malonic acid, succinic acid, fumaric acid, glucoheptonic acid, glucuronic acid, lactobioic acid, cinnamic acid, pyruvic acid, orotic acid, glyceric acid, glycyrrhizic acid, sorbic acid, hyaluronic acid, alginic acid, oxalic acid, salts thereof, and combinations thereof.

The organic carboxylic acid may be citric acid or a salt thereof.

The carboxylic acid may be or comprise a polymeric or polymerised carboxylic acid such as, for example, polyacrylic acid, polymethacrylic acid, a copolymer of acrylic acid and methacrylic acid, polylactic acid, polyglycolic acid, or a copolymer of lactic acid and glycolic acid. The term “organic carboxylic acid” used herein also cover partial or full esters of organic carboxylic acids or partial or full salts thereof, provided that those can serve as a proton source in use according to the present invention.

The organic non-carboxylic reducing acid may, for example, be selected from ascorbic acid; ascorbate palmitic acid (ascorbyl palmitate); ascorbate derivatives such as 3-O-ethyl ascorbic acid, other 3-alkyl ascorbic acids, 6-O-octanoyl ascorbic acid, 6-O-dodecanoyl ascorbic acid, 6-O-tetradecanoyl ascorbic acid, 6-O-octadecanoyl ascorbic acid and 6-O-dodecanedioyl ascorbic acid; acidic reductones such as reductic acid; erythorbic acid; salts thereof; and combinations thereof.

The organic non-carboxylic reducing acid may be ascorbic acid or a salt thereof.

The one or more organic carboxylic acid or organic non-carboxylic reducing acid of the proton source may suitably be present with the conjugate base thereof. The acid and its conjugate base may suitably form a buffer when contacted with or exposed to an aqueous environment. The acid and its conjugate base may be provided in a ratio to achieve the desired pH upon exposure to an aqueous environment.

The buffer system may be selected so that a desired pH is achieved upon exposure to an aqueous environment and maintained as the NOx generating reaction proceeds. The buffer system may be selected so that pH of the reaction may be in the range of about 3 to 9, for example about 4 to 8. For physiological contact or for contact with living cells and organisms, the pH of the reaction may be in the range of about 5 to about 8. The conjugate base, where present, may be added separately, or may be generated in situ from the proton source by adjustment of the pH using an acid and/or base, for example a mineral acid and/or a mineral base.

The proton source may be a citric acid/citrate buffer system, for example and citric acid/trisodium citrate buffer system.

It is understood by the skilled person that the choice of acid component/proton source may be selected depending on the desired use of the solid powder composition.

### Particles in the Solid Powder Composition

The solid powder composition may include particles containing both a nitrite salt and a proton source. In other words, the solid composition includes particles wherein one or more of the particles contain the proton source and the nitrite salt within the same particle. In this way, the proton source component and nitrite salt component may be held in close proximity even when the powder is dispersed (e.g., through inhalation of the powder).

The particles of the solid composition may be a suitable particle size for their desired use or application. For example, the particles of the solid composition may have a particle size of about 10 μm or less, for example, about 5 μm or less, about 4 μm or less, about 3 μm or less, about 2 μm or less or about 1 μm or less.

Alternatively, the particles of the solid composition may have a particle size of greater than 5 μm. For example, the particles of the solid composition may have a particle size of greater than 50 μm, greater than 100 μm, greater than 250 μm, greater than 500 μm, greater than 750 μm, greater than 1000 μm.

The weight ratio of nitrite to proton source in the solid composition may be in the range of about 1:1 to about 1:99, such as in the range of about 1:4 to about 1:49 or about 1:7 to about 1:24.

The solid powder composition may comprise further optional additives, such as a binding agent or an organic polyol.

### Binding Agent

The solid powder composition may be substantially free of one or more binding agents. Alternatively, the solid powder composition may further include one or more binding agents. A “binding agent” used herein refers to an agent that promotes the adhesion of particles.

Suitable binding agents may include sugars, natural binders or synthetic or semisynthetic polymer binders. Sugar species may include, for example, sucrose or liquid glucose. Natural binders may include, for example, acacia, tragacanth, gelatin, starch paste, pregelatinized starch, alginic acid or cellulose. Synthetic or semisynthetic polymer binders may include, for example, methyl cellulose, ethyl cellulose, hydroxy propyl methyl cellulose (HPMC), hydroxy propyl cellulose, sodium carboxy methyl cellulose, polyvinylpyrrolidones (PVP), polyethylene glycols (PEG), polyvinyl alcohols, polymethacrylates. The binding agent may be a copolymer of 1-vinyl-2-pyrrolidone and vinyl acetate (copovidone). The binding agent may be microcrystalline cellulose.

The binding agent may be incorporated into the composition in % w/w of about 5% w/w to about 30% w/w. For example, the binding agent may be incorporated into the composition in a % w/w of about 10% w/w to about 25% w/w.

### Organic Polyol

The solid powder composition may be substantially free of one or more organic polyols. Alternatively, the solid powder composition may further include one or more organic polyol. When the solid powder composition includes one or more organic polyols, it is preferred that the organic polyol is added to the composition after any processing which involves removal of solvent (e.g., after spray drying or lyophilisation steps). In other words, the polyol may be added to a composition including one or more particles containing a nitrite salt and a proton source.

The expression “organic polyol” herein refers to an organic molecule with two or more hydroxyl groups that is not a proton source, particularly for a nitrite salt reaction, and is not a saccharide or polysaccharide (the terms “saccharide” and “polysaccharide” include oligosaccharide, glycan and glycosaminoglycan). The organic polyol will thus have a pKa₁ of about 7 or greater.

The expression “organic polyol” herein preferably excludes reductants. Examples of reductants which are organic molecules with two or more hydroxyl groups and not a saccharide or polysaccharide are thioglycerol (for example, 1-thioglycerol), hydroquinone, butylated hydroquinone, ascorbic acid, ascorbate, erythorbic acid and erythorbate. Thioglycerol (for example, 1-thioglycerol), hydroquinone, butylated hydroquinone, ascorbate and erythorbate are thus preferably excluded from the expression “organic polyol” because they are reductants. Ascorbic acid and erythorbic acid are excluded from the expression anyway because they are proton sources, particularly for the nitrite salt reaction.

The organic polyol may be cyclic or acyclic or may be a mixture of one or more cyclic organic polyol and one or more acyclic organic polyol. For example, the one or more organic polyol may be selected from one or more alkane substituted by two or more OH groups, one or more cycloalkane substituted by two or more OH groups, one or more cycloalkylalkane substituted by two or more OH groups, and any combination thereof. The organic polyol may not carry any substituents other than OH.

The one or more organic polyol may be one or more acyclic organic polyol. The one or more acyclic organic polyol may be selected from the sugar alcohols having 4, 5, 6, 7, 8, 9, 10, 11 or 12 carbon atoms. The one or more acyclic organic polyol may be selected from the alditols, for example the alditols having 4, 5, 6, 7, 8, 9, 10, 11 or 12 carbon atoms. The one or more organic polyol may not include a saponin, sapogenin, steroid or steroidal glycoside.

Alternatively, the one or more organic polyol may be one or more cyclic organic polyol. The one or more cyclic organic polyol may be a cyclic sugar alcohol or a cyclic alditol. For example, the one or more cyclic polyol may be a cyclic sugar alcohol having 4, 5, 6, 7, 8, 9, 10, 11 or 12 carbon atoms or a cyclic alditol having 4, 5, 6, 7, 8, 9, 10, 11 or 12 carbon atoms. A specific example of a cyclic polyol is inositol.

The one or more organic polyol may have 7 or more hydroxy groups. The one or more organic polyol may be a sugar alcohol or alditol having 7 or more hydroxy groups. The one or more organic polyol may have 9 or more hydroxy groups. The one or more organic polyol may be a sugar alcohol or alditol having 9 or more hydroxy groups. The one or more organic polyol may have 20 or fewer hydroxyl groups. The one or more organic polyol may be a sugar alcohol or alditol having 20 or fewer hydroxy groups. The one or more organic polyol may have 15 or fewer hydroxyl groups. The one or more organic polyol may be a sugar alcohol or alditol having 15 or fewer hydroxyl groups. The one or more organic polyol may have a number of hydroxyl groups in the range of 7 to 20, for example, in the range of 9 to 15. The one or more organic polyol may include 9, 12, 15 or 18 hydroxy groups.

The one or more organic polyol may be a sugar alcohol compound comprising, for example consisting of, one or more monosaccharide units and one or more acyclic sugar alcohol units. The one or more organic polyol may be a sugar alcohol compound comprising, for example consisting of, a straight chain of one or more monosaccharide units and one or more acyclic sugar alcohol units or a branched chain of one or more monosaccharide units and one or more acyclic sugar alcohol units.

A “monosaccharide unit” as used herein refers to a monosaccharide covalently linked to at least one other unit (whether another monosaccharide unit or an acyclic sugar alcohol unit) in the compound. An “acyclic sugar alcohol unit” as used herein refers to an acyclic sugar alcohol linked covalently to least one other unit (whether a monosaccharide unit or another acyclic sugar alcohol unit) in the compound. The units in the compound may be linked through ether linkages. One or more of the monosaccharide units may be covalently linked to other units of the compound through a glycosidic bond. Each of the monosaccharide units may be covalently linked to other units of the compound through a glycosidic bond. The sugar alcohol compound may be a glycoside with a monosaccharide or oligosaccharide glycone and an acyclic sugar alcohol aglycone.

Acyclic sugar alcohol units may be sugar alcohol units having 4, 5, 6, 7, 8, 9, 10, 11 or 12 carbon atoms. The acyclic sugar alcohol unit may be selected from the group consisting of units of erythritol, threitol, arabitol, xylitol, ribitol, mannitol, sorbitol, galactitol, fucitol, iditol and volemitol.

One or more of the monosaccharide units may be a C₅ or C₆ monosaccharide unit, i.e., a pentose or hexose unit. Each monosaccharide unit may be a C₅ or C₆monosaccharide unit. One or more of the sugar alcohol units may be a C₅ or C₆ sugar alcohol unit. Each sugar alcohol unit may be a C₅ or C₆ sugar alcohol unit.

The sugar alcohol compound may comprise, for example may consist of, n monosaccharide units and m acyclic sugar alcohol units, where n is a whole number and at least one, m is a whole number and at least one and (n+m) is no more than 10. The sugar alcohol compound may comprise, for example may consist of, a chain of n monosaccharide units terminated with one acyclic sugar alcohol unit, where n is a whole number between one and nine. The chain of monosaccharide units may be covalently linked by glycosidic bonds. Each monosaccharide unit may be covalently linked to another monosaccharide unit or the acyclic sugar alcohol unit by a glycosidic bond. The sugar alcohol compound may comprise, for example may consist of, a chain of 1, 2 or 3 monosaccharide units terminated with one acyclic alcohol unit. 1, 2, 3 or each monosaccharide unit may be a C₅ or C₆ monosaccharide unit. The acyclic alcohol unit may be a C₅ or C₆ sugar alcohol unit. Examples of the sugar alcohol compound include but are not limited to: isomalt, maltitol and lactitol (n=1); maltotriitol (n=2); and maltotetraitol (n=3).

Such sugar alcohol compounds may be described as sugar alcohols derived from a disaccharide or an oligosaccharide. “Oligosaccharide”, as used herein, refers to a saccharide consisting of three to ten monosaccharide units. Sugar alcohols derived from disaccharides or oligosaccharides may be synthesised (e.g. by hydrogenation) from disaccharides, oligosaccharides or polysaccharides (e.g. from hydrolysis and hydrogenation), but are not limited to compounds synthesised from disaccharides, oligosaccharides or polysaccharides. For example, sugar alcohols derived from a disaccharide may be formed from the dehydration reaction of a monosaccharide and a sugar alcohol. The one or more organic polyol may be a sugar alcohol derived from a disaccharide, trisaccharide or tetrasaccharide. Examples of sugar alcohols derived from disaccharides include but are not limited to isomalt, maltitol and lactitol. An example of a sugar alcohol derived from a trisaccharide includes but is not limited to maltotriitol. An example of a sugar alcohol derived from a tetrasaccharide includes but is not limited to maltotetraitol.

Organic polyols may be selected from erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, and any combination thereof. Glycerol can be used, and when present is preferably in association with one or more other organic polyol, for example erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, or any combination thereof.

Many organic polyols contain one or more chiral centre and thus exist in stereoisomeric forms. All stereoisomeric forms and optical isomers and isomer mixtures of the organic polyols are intended to be included within the scope of this invention. In particular, the D and/or L forms of all chiral organic polyols and all mixtures thereof may be used.

### Coated Particles

The solid powder composition may include particles coated with a hydrophobic material (also referred to herein as coated particles).

The coated particles may include a single particle containing a nitrite salt and a proton source and coated with the hydrophobic material.

In this way, the coated particles include nitrite salt and proton source within the same coating.

The hydrophobic material may be any material capable of coating the particles such that the particles are coated with a hydrophobic layer. The hydrophobic material may be a polymeric material, for example an organic polymeric material. The hydrophobic material may be an amphiphilic species, for example, a surfactant-type species such as a non-ionic, anionic, cationic or amphoteric surfactant-type species. The hydrophobic material may be an inorganic mineral material, for example, and inorganic mineral material that forms a 3D framework. The hydrophobic material may be biocompatible. The hydrophobic material may include one or more of poly (lactic-co-glycolic acid) (PLGA), dipalmitoylphosphatidylcholine (DPPC), magnesium stearate, and mesoporous silica. The hydrophobic material may comprise the polymeric material poly (lactic-co-glycolic acid) (PLGA) without an acid end group or may comprise the polymeric material poly (lactic-co-glycolic acid) (PLGA) with an acid end group.

A “surfactant” as used herein refers to a surface-active agent which can lower the surface tension of a species in a medium or the interfacial tension between mediums. Surfactant species generally have a hydrophilic head and a hydrophobic tail.

The hydrophobic material may adhere to the particles by chemical bonding or by electrostatic or intermolecular forces.

The coating of the coated particles or may affect the reaction dynamics, for example the reaction kinetics, of the acidification of the nitrite salt when the coated particles are exposed to an aqueous environment.

The coated particles of the solid composition may be a suitable particle size for the desired use or application. The coated particles of the solid composition may have a particle size of about 10 μm or less, for example, about 5 μm or less, about 4 μm or less, about 3 μm or less, about 2 μm or less or about 1 μm or less. Alternatively, the coated particles of the solid composition may have a particle size of greater than about 5 μm. For example, the particles of the solid composition may have a particle size of greater than about 50 μm, greater than about 100 μm, greater than about 250 μm, greater than about 500 μm, greater than about 750 μm, greater than about 1000 μm.

Forming the Particles from a Mixture Containing a Nitrite Salt Solution and a Proton Source Solution

The particles of the solid powder composition may be formed from a mixture containing a nitrite salt solution and a proton source solution. Particles formed in this way should be formed by removal of solvent in a short time (e.g., thirty seconds or less) after mixing the nitrite salt solution and the proton source solution and/or the mixture is placed under reaction retarding conditions (e.g. at a temperature less than the freezing point of the solvent) after mixing nitrite salt solution and the proton source solution and for solvent removal. In this way, the solvent is removed from the mixture while minimising the acidification of the nitrite. An effective amount of nitrite and proton source may therefore be present in the resulting powder composition.

When the solvent is removed in a short time after mixing the nitrite salt solution and the proton source solution, the solvent may be removed in thirty second or less after the nitrite solution and proton source solution is mixed. In some examples, the solvent is removed in ten seconds or less, five seconds or less, two seconds or less or one second or less after mixing the nitrite solution and the proton source solution. In some examples, the solvent is removed in 500 milliseconds or less, 100 milliseconds or less, 50 milliseconds or less or 10 milliseconds or less after mixing the nitrite solution and the proton source solution.

In one example, the particles may be formed by spray-drying a mixture containing a nitrite salt solution and a proton source solution. Spray-drying of the mixture may allow the removal of solvent in a time of thirty seconds or less after mixing of the nitrite salt solution and the proton source solution. Spray-drying of materials is known per se.

The mixture is typically a mixture of an aqueous solution of the nitrite salt and an aqueous solution of the proton source. When aqueous solutions are used, the time between mixing the two aqueous solutions is minimised to suppress acidification of the nitrite salt. The aqueous solution of the nitrite salt and the aqueous solution of the acid may be mixed in line for about 1 to about 10 milliseconds, for example about 3 to about 5 milliseconds, before spray-drying takes place. Spray-drying may occur immediately after mixing of the nitrite and acid solutions. It is understood that mixing and spray-drying a mixture containing a nitrite salt solution and a proton source solution, as described, limits the potential reaction time between the proton source and nitrite component.

The particles formed by spray-drying the mixture containing a nitrite salt solution and an acid solution may have a particle size of about 10 μm or less, for example, about 5 μm or less, about 4 μm or less, about 3 μm or less, about 2 μm or less, or about 1 μm or less.

Spray-drying a mixture containing a nitrite salt solution and an acid solution as described may result in a solid powder composition where each particle contains nitrite salt and proton source components.

Particles formed by spray-drying a mixture containing a nitrite salt solution and a proton source solution may be any suitable morphology. For example, particles formed by spray-drying a mixture containing a nitrite salt solution and proton source solution may be crystalline in form or amorphous in form. The particles formed by spray-drying a mixture containing a nitrite salt solution and a proton source solution may be amorphous in form.

Additionally or alternatively, the mixture of nitrite salt solution and proton source solution is placed under a reaction-retarding condition (e.g. at a temperature less than the freezing point of the solvent) before, during or immediately after mixing the nitrite salt solution and the proton source solution and for solvent removal. In this way, the acidification of the nitrite is retarded until the solvent is removed. In particular, the solvent may be an aqueous solvent.

A particular example of a reaction-retarding condition is a temperature of the mixture below the freezing point of the solvent. In this way, the reaction rate of the acidification of nitrite may be slowed while the solvent is removed. Where the temperature of the mixture is below the freezing point of the solvent, the nitrite solution and the proton source solution are typically mixed at a temperature above the freezing point of the solvent before the temperature of the mixture is reduced to below the freezing point of the solvent. In this way, good mixing of the solutions may occur.

In some examples, the solvent removal may occur at a reduced gas pressure. In particular, the solvent removal may occur at a reduced gas pressure in combination at a temperature below the freezing point of the solvent to be removed.

A particularly useful technique to remove the solvent under a reaction-retarding condition is lyophilisation (also referred to as “freeze-drying”).

It should be noted that the terms “removal of solvent” and/or “drying” as used herein to achieve a solid powder composition. These terms include but are not limited to the complete removal of solvent. In some examples, a solid powder composition may include trace amounts of residual solvent. For example, the powder composition may contain up to about 10% of residual solvent, for example up to about 5% residual solvent, up to about 3% residual solvent or up to about 1% residual solvent. Additional drying techniques, such as vacuum drying, may be employed after the initial removal of solvent in order to provide the solid powder composition.

### Pharmaceutical Compositions

The solid powder compositions disclosed herein may be included in a pharmaceutical composition, optionally with one or more pharmaceutically acceptable carriers, excipients and/or adjuvants. Such carriers, excipients and/or adjuvants may be physiologically compatible when desired for use in vivo.

Examples of carriers and/or excipients, for example carriers and or excipients that are physiologically compatible, include without limitation lactose, starch, dicalcium phosphate, magnesium stearate, sodium saccharin, talcum, cellulose, cellulose derivatives, sodium crosscarmellose, glucose, gelatin, sucrose, magnesium carbonate, magnesium chloride, magnesium sulfate, calcium chloride and the like.

Generally speaking, depending on the intended mode of administration, the pharmaceutical composition will contain about 0.005% to about 95%, preferably about 0.5% to about 50% by weight of the combination or composition of the present invention or components thereof. Actual methods of preparing such dosage forms are known, or will be apparent to those skilled in the art.

Excipients may be selected from known excipients depending on the intended use or administration route whereby the reactants and/or reaction products are to be delivered to the target site for the delivery of the nitric oxide, optionally other oxides of nitrogen and/or optionally precursors thereof. For example, creams, lotions and ointments may be formulated by incorporating the nitrite salt into excipients such as cream, lotion and ointment bases or other thickening agents and viscosifying agents (for example Eudragit L100, carbopol, carboxymethylcellulose or hydroxymethylcellulose). The proton source may be incorporated into excipients selected from carbopol, carboxymethylcellulose, hydroxymethylcellulose, methylcellulose, ethanol, lactose or in an aqueous base. If it is desired to form a film, film forming excipients such as, for example, propylene glycol, polyvinylpyrrolidone (povidone), gelatin, guar gum and shellac may be used.

Optional additional components may, for example, be selected from sweetening agents, taste-masking agents, thickening agents, viscosifying agents, wetting agents, lubricants, binders, film-forming agents, emulsifiers, solubilising agents, stabilising agents, colourants, odourants, salts, coating agents, antioxidants, pharmaceutically active agents and preservatives. Such components are well known in the art and a detailed discussion of them is not necessary for the skilled reader. Examples of auxiliary substances such as wetting agents, emulsifying agents, lubricants, binders, and solubilising agents include, for example, sodium phosphate, potassium phosphate, gum acacia, polyvinylpyrrolidone, cyclodextrin derivatives, sorbitan monolaurate, triethanolamine acetate, triethanolamine oleate and the like. A sweetening agent or a taste-masking agent may, for example, include a sugar, saccharin, aspartame, sucralose, neotame or other compound that beneficially affects taste, after-taste, perceived unpleasant saltiness, sourness or bitterness, that reduces the tendency of an oral or inhaled formulation to irritate a recipient (e.g. by causing coughing or sore throat or other undesired side effect, such as may reduce the delivered dose or adversely affect patient compliance with a prescribed therapeutic regimen). Certain taste-masking agents may form complexes with one or more of the nitrite salts. Examples of thickening agents, viscosifying agents and film-forming agents have been given above.

Examples of pharmaceutically active agents that may be incorporated in the components and compositions or co-administered with the components and compositions according to the present invention include antibiotics, steroids, anaesthetics (for example topical anaesthetics such as lignocaine (lidocaine), amethocaine (tetracaine), xylocaine, bupivacaine, prilocaine, ropivfacaine, benzocaine, mepivocaine, cocaine or any combination thereof), analgesics, anti-inflammatory agents (for example non-steroidal anti-inflammatory drugs (NSAIDs)), anti-infective agents, vaccines, immunosuppressants, anticonvulsants, anti-dementia drugs, prostaglandins, antipyretics, anticycotics, anti-psoriasis agents, antiviral agents, vasodilators or vasoconstrictors, sunscreen preparations (e.g. PABA), antihistamines, hormones such as oestrogen, progesterone or androgens, antiseborrhetic agents, cardiovascular treatment agents such as alpha or beta blockers or Rogaine, vitamins, skin softeners, enzymes, mast cell stabilizers, scabicides, pediculicides, keratolytics, lubricants, narcotics, shampoos, anti-acne preparations, burn treatment preparations, cleansing agents, deodorants, depigmenting agents, diaper (nappy) rash treatment products, emollients, moisturizers, photosensitizing agents, poison ivy or poison oak or sumac products, sunburn treatment preparations, proteins, peptides, proteoglycans, nucleotides, oligonucleotides (such as DNA, RNA, etc), minerals, growth factors, tar-containing preparations, honey-containing preparations (for example, preparations containing Manuka honey), wart treatment preparations, wet dressings, wound care products, or any combination thereof.

Particular examples include analgesic agents, such as ibuprofen, indomethacin, diclofenac, acetylsalicylic acid, paracetamol, propranolol, metoprolol, and oxycodone; thyroid release hormone; sex hormones, such as oestragen, progesterone and testosterone; insulin; verapamil; vasopressin; hydrocortisone; scopolamine; nitroglycerine; isosorbide dintirate; anti-histamines, such as terfenadine; clonidine; nicotine; non-steroidal immunosuppressant drugs, such as cyclosporine, methotrexate, azathioprine, mycophenylate, cyclophosphamide, TNF-α antagonists and anti-IL5, -IL4Ra, -IL6, -IL13, -IL17, -IL23 cytokine monoclonal antibodies; anti-convulsants; and drugs for Alzheimer's, dementia and/or Parkinson's disease, such as apamorphine and rivastigmine.

If optional additives are added to the pharmaceutical composition comprising the solid powder composition disclosed herein, these optional additives may be in the solid-state, for example in dry particulate form.

### Methods of Producing Solid Powder Compositions

### Method of Producing the Solid Powder Composition by Solvent Removal

The method of making the solid powder composition may include removing solvent from a mixture of a nitrite solution and a proton source solution in such a way so as to minimise acidification before the powder compositions forms.

In one example, the method includes the step of removing the solvent in less than thirty seconds (e.g. by spray-drying) after mixing of a nitrite solution and a proton source solution to form the solid.

In another example, the method includes providing reaction-retarding conditions (e.g. lyophilisation) and during solvent removal and before, during and/or immediately after mixing a nitrite salt solution and a proton source solution.

In one example, the method may include the step of removing the solvent from an aqueous mixture containing a nitrite salt solution and a proton source solution to form the solid powder.

The aqueous solution of the nitrite salt may have a concentration in the range of about 0.1 M to about 5 M. The aqueous solution of the nitrite salt may have a concentration of at least about 0.1 M, at least about 0.2 M, at least about 0.5 M, at least about 0.75 M, or at least about 1 M. The aqueous solution of the nitrite salt may have a concentration of up to about 5 M, up to about 4 M, up to about 3 M or up to about 2 M. For example, the aqueous solution of the nitrite salt may have a concentration in the range of about 1 M to about 2 M, such as about 1.5 M. The aqueous solution of the nitrite salt may have a pH of about 6.5 to about 9, for example, from about 7 to about 8.

The aqueous solution of the proton source may have a concentration in the range of about 0.1 M to about 5 M. The aqueous solution of the nitrite salt may have a concentration of at least about 0.1 M, at least about 0.2 M, at least about 0.5 M, at least about 0.75 M, or at least about 1 M. The aqueous solution of the nitrite salt may have a concentration of up to about 5 M, up to about 4 M, up to about 3 M or up to about 2 M. For example, the aqueous solution of the nitrite salt may have a concentration in the range of about 0.5 M to about 1.5 M, such as about 1 M. The aqueous solution of the citric acid may have a pH of about 4 to 6. The pH of the aqueous solution of the proton source may be adjusted using, for example a mineral base such as sodium hydroxide.

In some examples, the step of removing the solvent takes 20 seconds or less, ten seconds or less, five seconds or less, two seconds or less or one second or less after mixing the nitrite solution and the proton source solution. In some examples, the solvent is removed in 500 milliseconds or less, 100 milliseconds or less, 50 milliseconds or less or 10 milliseconds or less after mixing the nitrite solution and the proton source solution.

### Spray Drying

The solid powder compositions may be produced by spray-drying a nitrite solution and a proton source solution.

The aqueous solution of the nitrite salt and the aqueous solution of the acid may be mixed in line for about 1 to about 10 milliseconds, for example about 3 to about 5 milliseconds, before spray-drying takes place. Spray-drying may occur immediately after mixing of the nitrite and proton source solutions. It is understood that mixing and spray-drying a mixture containing a nitrite salt solution and a proton source solution, as described, greatly limits the potential reaction time between the proton source and nitrite component and halts the reaction entirely upon the rapid removal of moisture.

The spray-drying may occur at an outlet temperature in the range of about 60 to about 80° C., such as about 65 to about 75° C. or about 68 to about 70° C. The spray-drying may occur at an atomisation pressure in the range of about 1 to 6 bar. The spray-drying may occur at a liquid feed rate in a range of about 1 to about 5 g/min, such as about 2 g/min to about 4 g/m, or about 3 g/min.

### Reaction-Retarding Condition(s)

As an alternative, the method may include providing reaction-retarding conditions (e.g. lyophilisation) and during solvent removal and before, during and/or immediately after mixing a nitrite salt solution and a proton source solution.

A particular example of a reaction-retarding condition is a temperature of the mixture below the freezing point of the solvent. In this way, the reaction rate of the acidification of nitrite may be slowed while the solvent is removed. Where the temperature of the mixture is below the freezing point of the solvent, the nitrite solution and the proton source solution are typically mixed at a temperature above the freezing point of the solvent before the temperature of the mixture is reduced to below the freezing point of the solvent. In this way, good mixing of the solutions may occur.

In some examples, the solvent removal may occur at a reduced gas pressure. In particular, the solvent removal may occur at a reduced gas pressure in combination at a temperature below the freezing point of the solvent to be removed.

A particularly useful technique to remove the solvent under a reaction-retarding condition is lyophilisation (also referred to as “freeze-drying”).

The time taken to remove solvent after mixing the nitrite solution and the proton source solution under the retarded-reaction conditions may be about 10 minutes or less. Under these conditions, it may be less important to remove the solvent (e.g. water) so rapidly.

However, removal of solvent in a relatively short time frame is also desired to further limit acidification of the nitrite. In some examples, the solvent is removed under reaction-retarding conditions in about 8 minutes or less, for example, about 7 minutes or less, about 6 minutes or less, about 5 minutes or less, about 4 minutes or less, about 3 minutes or less or about 2 minutes or less after mixing the nitrite solution and the proton source solution. In further examples, the step of removing the solvent takes about 1 minute or less, about 30 seconds or less, about 20 seconds or less, about 15 seconds or less or about 10 second or less after mixing the nitrite solution and the proton source solution.

It should be noted that the terms “removal of solvent” and/or “drying” as used herein to achieve a solid powder composition. These terms include but are not limited to the complete removal of solvent. In some examples, a solid powder composition may include trace amounts of residual solvent. For example, the powder composition may contain up to about 10% of residual solvent, for example up to about 5% residual solvent, up to about 3% residual solvent or up to about 1% residual solvent. Additional drying techniques, such as vacuum drying, may be employed after the initial removal of solvent in order to provide the solid powder composition.

Methods of Producing Solid Powder Compositions with Coated Particles

A solid powder composition may be produced comprising particles coated in a hydrophobic material. The method may include the step of coating particles containing a nitrite salt and a proton source with a hydrophobic material.

The hydrophobic material may be the same hydrophobic material as described above.

The particles may be coated in any suitable manner known to the person of skill in the art.

The particles may be coated by dispersing the particles in a solution containing a hydrophobic material and drying the solution to provide particles that are coated with a layer of the hydrophobic material. In some examples, the solution includes a non-polar solvent. In particular examples, the solution is free of polar solvent (e.g. methanol). Such polar solvents may dissolve at least part of the particle. In particular, the solution may be aqueous-free.

The hydrophobic material may, for example, be PLGA. The particles may be dried at a 1:1 w/w ratio with the hydrophobic material. The solution which the particles are dispersed or suspended in may be a solution of DCM and the hydrophobic material.

In particular embodiments, the suspension of particles in the hydrophobic material solution is dried by spray drying. The solution containing the hydrophobic material in which the particles are dispersed in may be spray-dried at an outlet temperature of about 28 to 30° C. The solution containing the hydrophobic material which the particles are dispersed in may be spray-dried at an atomisation pressure of about 1 bar. The solution containing the hydrophobic material which the particles are dispersed in may be spray-dried a liquid feed rate of about 2 g/min.

The coated particles may have a particle size of less than about 10 μm, for example less than about 9 μm, for example less than about 8 μm, less than about 7 μm, less than about 6 μm, or less than about 5 μm.

The particles may be coated by blending the particles with the hydrophobic material to provide particles that are coated with a layer of the hydrophobic material. The hydrophobic material may, for example, be DPPC, magnesium stearate, mesoporous silica or combinations thereof. The particles may be blended at a ratio of 1:1 w/w with the hydrophobic material. The hydrophobic material may be sieved prior to blending. Alternatively, the hydrophobic material may not be sieved prior to blending.

The particles may be blended with the hydrophobic material for a time of about 10 to about 40 minutes, or a time of about 15 to about 30 minutes.

### Aqueous Environment

The solid powder compositions of the present invention typically release NOx when in contact with an aqueous environment. The aqueous environment is not particularly limited.

The aqueous environment may be an aqueous biological fluid, such as a bodily fluid. Such bodily fluids may include wound discharge, airway surface liquid (such as respiratory mucus) and/or blood (such as blood plasma, blood serum).

Alternatively, the aqueous environment may be a sterile aqueous solution. The aqueous environment may be a saline solution.

In some embodiments the solid powder compositions may be sufficiently hygroscopic to absorb moisture from air, which is sufficient to start the release of NOx.

### Methods of Treatment or Prevention

### Methods of Treatment or Prevention of Respiratory Disease or Disorder

The present invention includes a method of treating or preventing a respiratory disease or disorder, the method includes the administration of a therapeutically effective amount of a solid powder composition or a pharmaceutical composition as disclosed herein.

In addition, the present invention provides a solid powder composition or a pharmaceutical composition as disclosed herein for use in a method of treating or preventing a respiratory disease or disorder.

The particles of the solid composition or pharmaceutical composition may have a particle size suitable for deep lung inhalation and respiratory applications. The particles in the solid powder composition may have an average particle size of 10 microns or less when used for the treatment or prevention of a respiratory disease or disorder. For example, the particles of the solid composition or pharmaceutical composition may have a particle size of about 10 μm or less, for example, about 5 μm or less, about 4 μm or less, about 3 μm or less, about 2 μm or less or about 1 μm or less.

The conditions treatable using the present invention may include lung diseases such as viral infections for example influenza, SARS-COV or SARS-COV-2, pulmonary arterial hypertension, ischemic reperfusion injury of the heart, brain and organs involved in transplantation, chronic obstructive pulmonary disease (COPD) (particularly, emphysema, chronic bronchitis), asthma including severe asthma and viral and bacterial induced exacerbations of asthma and refractory (non-reversible) asthma, intra-nasal or pulmonary bacterial infections such as pneumonia, tuberculosis, non-tuberculosis mycobacterial infections and other bacterial and viral lung infections, for example secondary bacterial infections following virus infections of the respiratory tract.

The property of nitric oxide to induce vasodilation characterises some of the treatments using the solid powder composition or pharmaceutical composition of the present disclosure and the NOx gas evolved therefrom.

A particular example of diseases, disorders and conditions responsive to vasodilation includes, but is not limited to conditions associated with ischaemia.

Conditions associated with tissue ischaemia include Raynauld syndrome, severe primary vasospasm, and tissue ischaemia, for example tissue ischaemia caused by surgery, septic shock, irradiation or a peripheral vascular disease (for example diabetes and other chronic systemic disease).

In some embodiments, the respiratory disease or disorder may be associated with the presence of one or more microbes in the subject to be treated. In other words, the respiratory disease or disorder may be associated with one or more microbial infections in the subject. The NOx gas evolved from the solid powder composition or pharmaceutical composition when exposed to an aqueous environment may have a biocidal or biostatic effect on a potentially wide range of microorganisms, leading to many anti-microbial treatments. The microbes may, for example, be any one or more selected from bacterial cells, viral particles and/or fungal cells, or microparasites, and may be individual cells, organisms or colonies.

When the microbe is present in a bacterial infection, a fungal infection, viral or microparasitic infection of a human or other animal, the infection may, for example, be in the context of a disease such as the common cold, influenza, tuberculosis, SARS, COVID-19, pneumonia or measles.

The bacterium may be a pathogenic bacterial species. The microbial infection may be an infection caused by a pathogenic bacterial species, including Gram positive and Gram negative, aerobic and anaerobic, antibiotic-sensitive and antibiotic-resistant bacteria.

Examples of bacterial species which may be targeted using the present invention include species of the Actinomyces, Bacillus, Bartonella, Bordetalla, Borrelia, Brucella, Campylobacter, Chlamydia, Chlamydophila, Clostridium, Corynebacterium, Enterococcus, Escherichia, Francisella, Haemophilus, Heliobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, or Yersinia genera. Any combination thereof can also be targeted by the present invention.

The microbe may be a pathogenic species of Corynebacterium, Mycobacterium, Streptococcus, Staphylococcus, Pseudomonas or any combination thereof.

The microbe to be targeted may be selected from Actinomyces israelii, Bacillus anthracis, Bacteroides fragilis, Bordetella pertussis, Borrelia burgdorferi, Borrelia garinii; Borrelia afzelii; Borrelia recurrentis; Brucella abortus; Brucella canis; Brucella melitensis; Brucella suis; Campylobacter jejuni; Chlamydia pneumoniae; Chlamydia trachomatis; Chlamydophila psittaci; Clostridium botulinum; Clostridium difficile; Clostridium perfringens; Clostridium tetani; Corynebacterium diphtheria; Ehrlichia canis; Ehrlichia chaffeensis; Enterococcus faecalis; Enterococcus faecium; Escherichia coli, such as Enterotoxigenic E. coli (ETEC), Enteropathogenic E. coli, Enteroinvasive E. coli (EIEC), and Enterohemorrhagic (EHEC), including E. coli O157: H7; Francisella tularensis; Haemophilus influenza; Helicobacter pylori; Klebsiella pneumoniae; Legionella pneumophila; Leptospira species; Listeria monocytogenes; Mycobacterium leprae; Mycobacterium tuberculosis; Mycobacterium abscessus; Mycobacterium ulcerans; Mycoplasma pneumoniae; Neisseria gonorrhoeae; Neisseria meningitides; Pseudomonas aeruginosa; Nocardia asteroids; Rickettsia rickettsia; Salmonella typhi; Salmonella typhimurium; Shigella sonnei; Shigella dysenteriae; Staphylococcus aureus; Staphylococcus epidermidis; Staphylococcus saprophyticus; Streptococcus agalactiae; Streptococcus pneumoniae; Streptococcus pyogenes; Streptococcus viridans; Treponema pallidum subspecies pallidum; Vibrio cholera; Yersinia pestis; and any combination thereof.

The microbe may be selected from Chlamydia pneumoniae, Bacillus anthracis, Corynebacterium diphtheria, Haemophilus influenza, Mycobacterium leprae, Mycobacterium tuberculosis, Mycobacterium abscessus, Mycobacterium ulcerans, Pseudomonas aeruginosa, Staphylococcus aureus, Streptococcus pneumoniae, or any combination thereof.

The microbe may be an antibiotic-resistant or antibiotic-sensitive pathogenic bacterial species or an antibiotic-resistant or antibiotic-sensitive strain of a bacterial species.

The use of nitric oxide to treat methicillin resistant Staphylococcus aureus (MRSA) and methicillin sensitive Staphylococcus aureus (MSSA) is described, for example, in WO 02/20026, the disclosure of which is incorporated herein by reference. An example of an antibiotic-resistant or antibiotic-sensitive pathogenic bacterial species which may be killed or treated using the present invention is thus methicillin resistant Staphylococcus aureus (MRSA) or methicillin sensitive Staphylococcus aureus (MSSA).

The microbe may be a pathogenic fungal species. The microbial infection may be an infection caused by a pathogenic fungal species, including pathogenic yeasts.

Examples of fungal species which may be targeted using the present invention include species of Aspergillus, Blastomyces, Candida (for example Candida auris), Coccidioides, Cryptococcus (in particular, Cryptococcus neofromans or Cryptococcus gattii), Hisoplamsa, Murcomycetes, Pneumocystis (for example Pneumocystis jirovecii), Sporothrix, Talaromyces, or any combination thereof.

Examples of fungal infections include aspergillosis (such as allergic bronchia pulmonary aspergillosis), tinea pedis (athlete's foot), infections caused by a pathogenic species of Candida, such as vaginal yeast infections, fungal toenail infections and diaper rash, tinea cruris (jock itch), and tinea corporis (ringworm).

The microbe may be a virus particle. The infection may be cause by a pathogenic virus.

Examples of viruses which may be targeted using the present invention include influenza viruses, parainfluenza viruses, adenoviruses, noroviruses, rotaviruses, rhinoviruses, coronaviruses, respiratory syncytial virus (RSV), astroviruses, and hepatic viruses. The compositions of the present invention may be used in the treatment or prevention of an infection caused by one of the group selected from H1N1 influenza virus, Infectious Bovine Rhinotracheitis virus, Bovine Respiratory Syncytial virus, Bovine Parainfluenza-3 virus, SARS-COV, SARS-COV-2, and any combination thereof.

The invention may be applied to treat a disease or disorder caused by a viral infection. Examples of such diseases which may be targeted by the present invention include respiratory viral diseases, gastrointestinal viral diseases, exanthematous viral diseases, hepatic viral disease, cutaneous viral diseases, hemorrhagic viral diseases, and neurological viral diseases.

Respiratory viral infections include influenza, rhinovirus (i.e. common cold virus), respiratory syncytial virus, adenovirus, coronavirus infections, for example, COVID-19, and severe acute respiratory syndrome (SARS). Gastrointestinal viral diseases include norovirus infections, rotavirus infections, adenovirus infections and astrovirus infections. Exanthematous viral diseases include measles, rubella, chickenpox, shingles, roseola, smallpox, fifth disease and chikungunya virus disease. Hepatic viral diseases include hepatitis A, hepatitis B, hepatitis C, hepatitis D and hepatitis E.

Cutaneous viral diseases include warts, such as genital warts, oral herpes, genital herpes and molluscum contagiosum. Hemorraghic viral diseases include Ebola, Lassa fever, denghue fever, yellow fever, Marbug hemorrhagic fever and Crimean-Congo hemorrhagic fever. Neurological viral diseases which may be targeted using the present invention include polio, viral meningitis, viral encephalitis and rabies.

The microbe may be a parasitic microorganism (microparasite). The infection may be caused by a pathogenic parasitic microorganism.

Examples of parasitic microorganisms which may be targeted using the present invention include protozoa.

In particular, the invention may target the protozoa groups of Sarcodina (e.g. amoeba, for example Entamoeba such as Entamoeba histolytica or Entamoeba dispar), Mastigophora (e.g. flagellates, for example Giardia and Leishmania), Ciliophora (e.g. ciliates, for example Balantidium), Sporozoa (e.g. Plasmodium and Cryptosporidium), and any combination thereof.

Parasitic infections that may be treated using the present invention include malaria, amoebic dysentery and leishmaniasis (e.g. cutaneous leishmaniasis, mucocutaneous leishmaniasis or visceral leishmaniasis).

In particular, the respiratory disease or disorder may be tuberculosis.

The subject may be an animal or human subject. The term “animal” herein generally can include human; however, where the term “animal” appears in the phrase “an animal or human subject” or the like, it will be understood from the context to refer particularly to non-human animals or that the reference to “human” merely particularises the option that the animal may be a human to avoid doubt.

The subject may be a human subject. The human subject may be an infant or adult subject.

The subject may be a vertebrate animal subject. The vertebrate animal may be in the Class Agnatha (jawless fish), Class Chondrichthyes (cartilaginous fish), Class Osteichthyes (bony fish), Class Amphibia (amphibians), Class Reptilia (reptiles), Class Aves (birds), or Class Mammalia (mammals). The subject may be an animal subject in the Class Mammalia or Aves.

The subject may be a domestic species of animal. The domestic species of animal may be one of:
commensals, adapted to a human niche (e.g., dogs, cats, guinea pigs)
prey or farm animals sought or farmed for food (e.g., cows, sheep, pig, goats); and
animals for primarily draft purposes (e.g., horse, camel, donkey)

Examples of domestic animals include, but are not limited to: alpaca, addax, bison, camel, canary, capybara, cat, cattle (including Bali cattle), chicken, collared peccary, deer (including fallow deer, sika deer, thorold's deer, and white-tailed deer), dog, donkey, dove, duck, eland, elk, emu, ferret, gayal, goat, goose, guinea fowl, guinea pig, greater kudu, horse, llama, mink, moose, mouse, mule, muskox, ostrich, parrot, pig, pigeon, quail, rabbit, rat (including the greater cane rat), reindeer, scimitar oryx, sheep, turkey, water buffalo, yak and zebu.

Incorporation or Encapsulation of the Solid Powder Composition into a Substrate

Herein is provided a material comprising a substrate and the solid powder composition disclosed herein, wherein particles of the solid powder composition are incorporated or encapsulated into the substrate. In this way, the solid powder composition may be held within the material by the substrate until exposure with moisture or an aqueous environment.

The substrate may be a synthetic or natural polymer species. The substrate may, for example, be polycaprolactone, polyurethane or polyacrylonitrile. The substrate may, for example, be cellulose.

The material may be a fibrous material comprising fibres of the substrate and particles of the solid powder composition incorporated or encapsulated into the fibrous material. Particles of the solid powder composition may be exposed or partially exposed on the surface of the substrate fibres or may be wholly encapsulated in the fibrous network and fibre cross-sections.

In some examples, the substrate is porous and at least some of the particles of the solid powder composition are in the pores of the substrate. In other words, the substrate may be porous and impregnated with particles of the solid powder composition. In some examples, the substrate is porous by including pores in the surface of the substrate. In other examples, the substrate may be a porous mesh of substrate elements, such as polymeric fibres, and the particles are in voids between the substrate elements. As a particular example, the particles of the solid powder composition may be impregnated into voids of a polymeric fibre mesh.

The particles of the solid composition may be a suitable particle size for dispersion in gelling fibres. The particles of the solid composition may have a particle size of greater than about 10 μm. For example, the particles of the solid composition may have a particle size of greater than about 50 μm, greater than about 100 μm, greater than about 250 μm, greater than about 500 μm, greater than about 750 μm, greater than about 1000 μm.

To achieve larger particle sizes, the particles may undergo granulation. “Granulation” refers to a process of combining particulate species to form larger particles known as granules. Granulation may occur, for example, by compressing the particles to provide tablets which can then be broken up into granules. The particles may be compressed at about 1 to about 10 MT (metric tonnes), for example, may be compressed at about 3 to about 7 MT. The particles may be compressed at about 3.8 MT. The particles may be compressed at about 6.5 MT. The tablets may be broken up into granules using a sieve, for example, a 1 mm sieve.

To promote compression, a binding agent may be added to the particles. Suitable binding agents may include sugars, natural binders or synthetic or semisynthetic polymer binders. Sugar species may include, for example, sucrose or liquid glucose. Natural binders may include, for example, acacia, tragacanth, gelatin, starch paste, pregelatinized starch, alginic acid or cellulose. Synthetic or semisynthetic polymer binders may include, for example, methyl cellulose, ethyl cellulose, hydroxy propyl methyl cellulose (HPMC), hydroxy propyl cellulose, sodium carboxy methyl cellulose, polyvinylpyrrolidones (PVP), polyethylene glycols (PEG), polyvinyl alcohols, polymethacrylates. The binding agent may be a copolymer of 1-vinyl-2-pyrrolidone and vinyl acetate (copovidone). The binding agent may be microcrystalline cellulose.

The binding agent may be incorporated into the composition in % w/w of about 5% w/w to about 30% w/w. For example, the binding agent may be incorporated into the composition in a % w/w of about 10% w/w to about 25% w/w.

Alternatively, the composition may be substantially free of binding agents.

Particle size may be increased by such means in order to ensure that the particles remain trapped (incorporated or encapsulated) between the fibres.

Herein is provided a method of incorporating or encapsulating the solid powder composition according to the first or second aspects into a substrate, the method includes the steps of (i) mixing the solid powder composition disclosed herein with a non-polar liquid containing the substrate or substrate precursor to form a liquid-particle mixture and (ii) solidifying the liquid-particle mixture to form a material incorporating or encapsulating the solid powder composition disclosed herein.

The liquid-particle mixture may be solidified by spinning the mixture into fibres. Techniques known to a person of skill in the art for the spinning of the fibres may be used. For example, the liquid-particle mixture may be solidified by dry spinning, wet spinning, gel spinning or electrospinning. The liquid-particle mixture may be solidified by electrospinning. “Electrospinning” refers to a fibre production method which uses electric force to draw charged threads of polymer solutions or polymer melts to fibre diameters. The liquid-particle mixture may be solidified by gel spinning. “Gel spinning” refers to a fibre production method which relies on temperature-induced physical gelation for solidification.

Alternatively, the particles of the solid powder may be incorporated into the substrate after the substrate has formed. For example, particles of the solid powder may be impregnated into a porous substrate, such as a fibrous mesh substrate. In these examples, the substrate is already formed and the solid powder composition is being added to it. A particular example of methods to impregnate solid powder compositions into porous substrates include those described in EP2331309 (and other techniques available from Fibroline France).

In some examples, the material as described above is a component of a topical dressing, e.g. a wound dressing. In alternative embodiments, the material may be or form part of a biologically implantable material or device. For example, the biologically implantable material or device may be a vascular and other stent, catheter, pacemaker, defibrillator, heart assist device, artificial valve, electrode, orthopaedic screw and pin, and another thin medical and/or implantable article.

### Coated Materials or Devices

Disclosed herein is a material or device, wherein the material or device includes a substrate and a spray-dried coating on an exterior surface of the substrate, the spray-dried coating being formed from spray-drying a mixture containing a nitrite salt solution and a proton source solution.

Disclosed herein is also a material or device, wherein the material or device includes a substrate and a coating on an exterior surface of the substrate, the coating being a homogenous solid containing a nitrite salt and a proton source.

As such, disclosed herein is a method of providing a material or a device, the method includes the step of spray-drying a mixture containing a nitrite salt solution and a proton source solution onto an exterior surface of a substrate to provide the material or device.

The material or device may be a topical dressing, for example, a wound dressing or bandage.

The material or device may be an inhaler (handheld and nebulizer).

The material or device may be a biologically implantable material or device. For example, the material or device may be a vascular and other stent, catheter, pacemaker, defibrillator, heart assist device, artificial valve, electrode, orthopaedic screw and pin, and another thin medical and/or implantable article.

Disclosed herein is a method of implanting a material or device as disclosed herein into a human or animal body.

The preferred or particular features disclosed above may be applied to each and every aspect of the present invention to the extent that such features and aspects are compatible.

### EXAMPLES

### Preparation of Solid Powder Compositions

### Materials and Analytical Methods

The following materials were obtained from commercial sources: sodium nitrite from Honeywell, citric acid from Sigma Aldrich, trisodium citrate from Merck, sodium hydroxide from Fisher, PLGA RG 502 H from Sigma Aldrich, mesoporous silica (Syloid 244FP) from Grace, dipalmitoyl phosphatidylcholine (DPPC) from Avanti, Kollidon VA64 Fine from BASF, microcrystalline cellulose from JRS Pharma and dichloromethane (DCM) from Sigma Aldrich. Deionised (DI) water (18.2 MQ) was prepared using an ELGA water purification system.

Unless stated otherwise, the following analytical methods were used.

### Dry Powder Particle Size Distribution (PSD) by Sympatec

Laser particle size analysis of spray dried powders was performed using a Sympatec HELOS particle size analyser equipped with an R3 lens (0.5-175.0 μm range)/R5 lens (0.5-875.0 μm range) and an ASPIROS dispersion unit. Dispersal was achieved using compressed air at a pressure of 3.00 bar and a depression of 60 mbar. ASPIROS glass tubes were filled with powder in a reduced humidity environment (<25% RH) and sealed with Parafilm until the measurement was taken. Measurements were made in triplicate unless stated and the mean data was reported.

### Example 1: Spray-Drying a Mixture Containing a Nitrite Salt Solution and a Proton Source Solution to Form the Solid Powder Composition

A feed solution of 1.5M sodium nitrite (feed solution 1) was prepared by dissolving the required sodium nitrite mass in deionised water. A feed solution of 1M citric acid (feed solution 2), adjusted to pH 4, was prepared by dissolving the required citric acid mass in deionised water and adjusting the pH to 4 using 10M aqueous sodium hydroxide solution. The pH of the solution was measured using a Mettler Toledo Seven Compact PH meter.

Feed solutions 1 and 2 were spray dried using a Buchi B290 spray dryer, fitted with a Buchi two-fluid nozzle. The two feed solutions were pumped simultaneously using separate feed lines (platinum-cured silicone L/S 14 tubing) connected using a Y-piece fitting and a single Masterflex peristaltic pump, which combined the feed solutions immediately prior to atomisation. A standard Buchi cyclone and collection pot were fitted for product collection.

The feed solutions were spray dried in two batches, with the following conditions:

| | Outlet | Atomisation | Liquid |
|---|---|---|---|
| | Temperature | pressure | Feed rate |
| Example | (° C.) | (bar) | (g/min) |
| 1A | 68-70 | 5.5 | 3.06 |
| 1B | 68-70 | 1.5 | 3.05 |

Both batches were then vacuum dried using an Edwards Super Modulyo freeze dryer set to 25° C. for 24 hours.

Particle size distribution measurements were then taken for both batches using a Sympatec HELOS particle size analyser equipped with an R3 lens (0.5-175.0 μm range) and a ASPIROS dispersion unit. Dispersal was achieved using compressed air at a pressure of 3.00 bar and a depression of 60 bar. Measurements were made in triplicated.

The resultant particles size distribution measurements were as follows:

Mean Particle size (n = 3)
by laser diffraction (μm)

| Example | X₁₀ | X₅₀ | X₉₀ | VMD |
|---|---|---|---|---|
| 1A | 1.27 | 2.92 | 5.96 | 3.32 |
| 1B | 1.83 | 3.84 | 7.54 | 4.35 |

VMD = volume mean diameter

### Reference Example 2: Micronizing a Nitrite Salt and Proton Source Separately, and then Blending to Product the Solid Composition

Sodium nitrite was micronised using an Atritor M3 fluid energy mill with a venturi pressure of 8 bar and grinding pressure of 2 bar. The sodium nitrite was fed directly into the hopper at a target feed rate of ˜2 g/min. The produced powder (component 4A) was collected into a single collection jar under reduced humidity (20% RH).

Citric acid and trisodium citrate were combined together in the following weight proportions: 16.51% and 83.49%, respectively. The mixture was blended at 47 rpm for 10 min using a Turbula T2F mixer.

The blend was micronised using an Atritor M3 fluid energy mill with a venturi pressure of 8 bar and grinding pressure of 2 bar. The blend was fed directly into the hopper at a target feed rate of ˜2 g/min. The produced powder (Component 4B) was collected into a single collection jar under reduced humidity (20% RH).

The micronised nitrite solid (component 2A) and the micronised citric acid solid (component 2B) were then blended in a ratio of 9:1 w/w citrate solid: nitrite solid, using a Turbula T2F mixer at 46 rpm for 20 minutes, resulting in the powder composition of Reference Example 2.

### NOx Evolution

Examples 1A and 2 were loaded into an APTAR Unidose nasal spray (https://www.aptar.com/products/pharmaceutical/uds/), which was supported in a rig 30 cm above a petri dish (9.8 cm diameter) containing agarose with Hanks' balanced salt solution and a pH indicator (phenol red). FIG. 1 shows the deposition pattern of the powder by virtue of localised pH modification by the particles where they land.

Immediately after application the plate was transferred into a sealed chamber and the oxides of nitrogen (NOx) were measured by Single lon Flow Tube Mass Spectrometry (SIFT-MS) over a period of 15 minutes. All powders, irrespective of their method of preparation, evolved nitric oxide. However, differences in the total quantity of NOx evolved are seen between the four powders over the course of fifteen minutes.

It should be noted that the agarose is buffered at neutral to slightly alkaline pH, which should inhibit the reaction, but the particles are able to overcome this buffering effect in the short term and counter-act the buffering in a localised area. The table below and FIG. 2 show the cumulative NO generation for Examples 1A and 2. The cumulative NO/nmols per mg of nitrite normalises the results of the experiments for the % of nitrite in the powder.

| | Cumulative | Cumulative NO/ |
|---|---|---|
| Example | NO/nmols | nmols · mgNitrite⁻¹ |
| EXAMPLE 1A | 2410 | 526 |
| EXAMPLE 2 | 562 | 120 |

### Coated Solid Powder Compositions

### Example 3: Particles Coated with Hydrophobic Materials DPPC or Mesoporous Silica

Example 1B was blended with mesoporous silica in a ratio of 1:1 w/w, using a Turbula T2F mixer at 46 rpm for 20 minutes, resulting in the powder composition of Example 3A.

Example 1B was blended with DPPC in a ratio of 1:1 w/w, using a Turbula T2F mixer at 46 rpm for 20 minutes, resulting in the powder composition of Example 3B.

### Example 4: Particles Coated with PLGA

A PLGA RG 502 H solution was prepared by dissolving 1.5 g of PLGA to about 30 mL of DCM to form a clear and colourless solution. 1.5 g Example 1B was added to this solution with stirring to form a 1:1 w/w ratio feed suspension as a visually uniform white suspension.

The feed suspension was spray-dried using a Buchi B290 spray dryer according to the method detailed above. Spray drying parameters are summarised below.

| | Outlet | Atomisation | Liquid |
|---|---|---|---|
| | Temperature | pressure | Feed rate |
| Example | (° C.) | (bar) | (g/min) |
| 4 | 28-29 | 1 | 2.0 |

In a reduced humidity environment (28% RH) sample vials were laid horizontally in individual weighing boats. The lids were removed and the openings were covered with foil with holes (pierced using a needle). Samples were transferred to an Edwards Super Modulyo freeze dryer set to 25° C. and vacuum dried for 24 h (maximum vacuum pressure observed was ˜0.1 mbar). Following vacuum drying, samples were transferred to a low humidity (˜24% RH) environment and overlaid with nitrogen. Vials were then sealed with Parafilm and sealed into foil pouches with desiccant for storage at 2-8° C.

Particle size distribution measurements were then taken using a Sympatec HELOS particle size analyser equipped with an R3 lens (0.5-175.0 μm range) and a ASPIROS dispersion unit. Dispersal was achieved using compressed air at a pressure of 3.00 bar and a depression of 60 bar. Measurements were made in triplicated.

The resultant particles size distribution measurements were as follows:

Mean Particle size (n = 3)
by laser diffraction (μm)

| Example | X₁₀ | X₅₀ | X₉₀ | VMD |
|---|---|---|---|---|
| 4 | 0.92 | 4.17 | 11.07 | 5.23 |

VMD = volume mean diameter

### Example 5: NOx Evolution of Coated Particles

An aliquot of the powder sample (30 mg) was deposited in a 60 mm petri dish. Cellulose filter paper (50 mm diameter) was placed over the top of the sample, and light pressure applied. Sodium phosphate solution (10 mM, 250 μl) was dispensed onto the cellulose filter paper. The sample was immediately placed into a 650 ml chamber, which was sealed, and then humified air was pulled through the chamber at 650 ml/min for thirty minutes. The air stream from the outfeed was analysed by Single Ion Flow Tube Mass Spectrometry (SIFT-MS).

| | | Average | Average cumulative |
|---|---|---|---|
| | | cumulative | NO/nmols per mg |
| Example | Description | NO/nmols | sodium nitrite |
| EXAMPLE 5A | Spray-dried powder of sodium | 22141 | 6044 |
| | nitrite (1.5M) and citric acid | | |
| | (1M, pH 4.0) with vacuum | | |
| | drying (as in Example 1) | | |
| EXAMPLE 5B | Powder of Example 5A spray- | 4806 | 3342 |
| | dried with PLGA RP 502H | | |
| | (1:1 w/w ratio) as per | | |
| | Example 4 | | |
| EXAMPLE 5C | Powder of Example 5A spray- | 4973 | 4382 |
| | dried with PLGA RP 503H | | |
| | (1:1 w/w ratio) as per | | |
| | Example 4 | | |
| EXAMPLE 5D | Powder of Example 5A | 4061 | 3010 |
| | blended with magnesium | | |
| | stearate (1:1 w/w ratio) | | |
| | as per Example 3 | | |
| EXAMPLE 5E | Powder of Example 5A | 4088 | 2786 |
| | blended with magnesium | | |
| | stearate (1:1 w/w ratio) | | |
| | as per Example 3 | | |
| EXAMPLE 5F | Powder of Example 5A | 7341 | 3294 |
| | blended with magnesium | | |
| | stearate (1:1 w/w ratio) | | |
| | as per Example 3 | | |

### Biological Assessment of Solid Powder Compositions

### Example 6: Assessment of the Efficacy of Four Formulations Against Pseudomonas aeruginosa

Petri dishes containing Nutrient Agar (NA, available from AcuMedia) were prepared and allowed to set. A Pseudomonas aeruginosa (ATCC 9027) inoculum was prepared in phosphate buffered saline (PBS, Sigma-Aldrich) and serially diluted to a final concentration of 1×10⁵ CFU mL⁻¹. 100 mL of inoculum was pipetted onto NA plates, spread, and allowed to dry at room temperature for 15 minutes. Lids were removed from the inoculated agar plates and the open plates were placed inside the Aptar Unidose nasal spray.

Aptar delivery devices containing either Example 1A, or Reference Example 2 powder were attached to the Aptar nasal spray devices and the powder was nebulized (approximately 50 mg dose) onto the agar plates. The table below shows the Examples used for each Formulation.

| | |
|---|---|
| Formulation 1 | Example 1A |
| Formulation 2 | Reference Example 2 |

After 5 seconds, the agar plate lids were replaced, and the agar plates were incubated for 16 hours at 37° C.±2° C. Following incubation, the plates were photographed. For all plates, three biopsy punches were taken from a 2×2 cm area in the centre of the agar plate. Sterile swabs moistened with PBS were used to remove the bacteria from each biopsy, any cells were suspended in 10 mL PBS before sonication for 5 minutes, serial dilution and were plated onto NA.

Negative control plates that were not exposed to nebulized powder, and positive control plates that had the addition of 1 mL bleach, were also tested concurrently. All testing was performed in quintuplicate.

For each test item, three replicates were randomly chosen, and DNA was extracted from 400 μL per biopsy using the DN easy Blood & tissue Kit (Qiagen), according to manufacturer's instructions. Samples were eluted in a final volume of 100 μL in AE buffer.

For each extraction, qPCR was performed in triplicate, using the QuantiNova Pathogen and IC kit (Qiagen) according to manufacturers instructions. Individual reaction tubes contained a final concentration of 16 μM for each primer and 5 μM labelled probes.

Cycle conditions were as follows: 50° C. for 10 min, 95° C. for 2 min, 35 cycles of 95° C. for 5 sec, 55° C. for 30 sec, 72° C. for 1 min. Each assay run was validated by positive (P. aeruginosa) and negative (RNase free water) controls. Data was analysed using the Q-Rex software (Qiagen) to obtain Cq values from a predetermined threshold value. For each sample, mean Cq values were compared to a standard curve with an established range of 1×10² to 1×10⁸ CFU mL⁻¹, to calculate final sample concentration in Log₁₀CFUmL⁻¹.

TABLE 1
Average recoveries and reductions of Pseudomonas aeruginosa of
three biopsy punches taken at the centre of nutrient agar seeded with 1 ×
10⁵ CFU mL⁻¹, following treatment with formulations 1 and 2 and
bleach compared to an untreated negative control (N = 5).

| | Average | Average | |
|---|---|---|---|
| | recovery ± SD | reduction ± SD | |
| | (Log₁₀CFU | (Log₁₀CFU | Statistical |
| Test Agents | mL⁻¹) | mL⁻¹) | significance |
| Negative Control | 7.44 ± 0.17 | N/A | N/A |
| Formulation 1 | 0.00 ± 0.00 | 7.44 ± 0.00 | *** |
| Formulation 2 | 1.36 ± 2.13 | 6.08 ± 2.13 | ** |
| Positive Control | 0.00 ± 0.00 | 7.44 ± 0.00 | *** |
| (bleach) | | | |

SD = standard deviation, CFU = colony forming units, N/A = not applicable, * = p < 0.05, ** = p < 0.01, *** = p < 0.001.

An average of P. aeruginosa recovery of 7.44±0.17 Log₁₀CFU mL⁻¹ was observed from biopsies taken from the negative control plate. Average P. aeruginosa recoveries of 1.36±2.13 Log¹⁰CFU mL⁻¹ was observed from biopsies taken from Formulation 2. No viable P. aeruginosa was recovered from biopsies taken from Formulation 1 or the positive control plate.

TABLE 2
Molecular quantification of P. aeruginosa of biopsy
punches taken from nutrient agar seeded with 1 × 10⁵ CFU
mL⁻¹, following treatment with formulation 1 and 2
and bleach compared to an untreated negative control.

| | Average Cq | Quantification ± SD | Statistical |
|---|---|---|---|
| Test Agents | Reading | (Log₁₀CFU mL⁻¹) | significance |
| Negative Control | 16.93 | 6.65 ± 0.18 | N/A |
| Formulation 1 | 33.91 | 1.15 ± 0.33^{#} | *** |
| Formulation 2 | 25.59 | 2.73 ± 1.18 | ** |
| Positive Control | 32.55 | 1.52^{~} | N/A |
| (bleach) | | | |

SD = standard deviation, CFU = colony forming units. ^{#} = Quantification was below the limit of detection. ^{~} = The quantification of the positive control samples was performed to N = 1 so no standard deviation could be calculated. N/A = not applicable, ** = p < 0.01, *** = p < 0.001.

Significant reductions in the recovery of viable P. aeruginosa were observed from biopsies taken from the nutrient agar plates seeded with a 1×10⁵ CFU mL⁻¹ inoculum following treatment with Formulation 1. Powders when compared to the untreated negative control, as no viable P. aeruginosa were recovered. Molecular quantification reflects the recovery from colony counts.

### Example 7: Effect of Powdered Compositions on the Sprouting of Human Umbilical Vein Endothelial Cells (HUVEC) in Spheroid-Based Cellular Angiogenesis Assay

10× concentrated stock solutions/suspensions of Examples 1B and 4A were prepared in basal medium (without supplement and FCS) by vortexing and pipetting. Subsequently, semi-log dilution series were prepared in the same medium.

| | Example 1B | Example 4A |
|---|---|---|
| # | [mg/mL] | [mg/mL] |
| 1 | 5.000 | 10.000 |
| 2 | 1.500 | 3.000 |
| 3 | 0.500 | 1.000 |
| 4 | 0.150 | 0.300 |
| 5 | 0.050 | 0.100 |
| 6 | 0.015 | 0.030 |
| 7 | 0.005 | 0.010 |

### Endothelial Cells

Cells: HUVEC, primary human umbilical vein endothelial cells (PromoCell, Heidelberg, Germany), passage 3 to 4.
Morphology: adherent, cobblestone-like growing as monolayer
Medium: endothelial cell growth and basal medium (ECGM/ECBM, PromoCell)
Subculture: split 1:3; every 3-5 days, seed out at ca. 1×10⁴ cells/cm²
Incubation: at 37°° C. with 5% CO₂
Doubling Time: 24-48 hours
Storage: frozen with 70% medium, 20% FCS, 10% DMSO at about 1×10⁶ cells/ampoule
Origin: human umbilical vein, pooled donors

### Test Method

The experiments were pursued in modification of the originally published protocol (Korff and Augustin: J Cell Sci 112:3249-58, 1999). In brief, spheroids were prepared as described (Korff and Augustin: J Cell Biol 143:1341-52, 1998) by pipetting 400 HUVEC in a hanging drop on plastic dishes to allow overnight spheroid aggregation. 50 HUVEC spheroids were then seeded in 0.9 ml of a collagen gel and pipetted into individual wells of a 24 well plate to allow polymerization. Preincubated test samples were added after 30 min by pipetting 100 μl of a 10-fold concentrated working solution on top of the polymerized gel (final assay concentrations see table 1). Plates were incubated at 37° C. for 24 hours and fixed by adding 4% PFA (Roth, Karlsruhe, Germany).

### Quantification

Sprouting intensity of HUVEC spheroids treated with the test samples were quantitated by an image analysis system determining the cumulative sprout length per spheroid (CSL). Pictures of single spheroids were taken using an inverted microscope and the digital imaging software NIS-Elements BR 3.0 (Nikon). Subsequently, the spheroid pictures were uploaded to the homepage of the company Wimasis for image analysis. The cumulative sprout length of each spheroid was determined using the imaging analysis tool WimSprout. The mean of the cumulative sprout length of 10 randomly selected spheroids was analyzed as an individual data point. Mean and SD values of each triplicate were converted into % of basal control.

### Result

FIG. 3 shows the CSL relative to the basal control of Examples 1B and 4A. The effect of Example 1B (spray-dried particles with no coating) is small compared to the basal control. In contrast, the PLGA-coated particles of Example 4A show a significant dose-dependent effect compared with the basal control. This indicates that the coated particles provide a localized environment that capable of the acidification of nitrite despite being in a substantially neutral environment.

### Example 8: SEM/EDX Study of Spray Dried Powders

SEM/EDX was used to assess the microstructure of the various components and their contribution in the final formulation of powders made in accordance with the method in Example 1.

### Experimental

The spray-dried powder was stored in a fridge prior to sampling. Initial preparation for the SEM/EDX study involved sprinkling the powders onto carbon adhesive discs on SEM specimen stubs. Subsequently, for clearer elemental mapping applications, a similar approach but with gentle compacting of the powder was employed.

In both cases, the prepared specimens were examined uncoated and in low vacuum mode using a FEI Quanta FEG 250 environmental SEM with associated Bruker Quantax 200 microanalysis system for elemental analysis.

### Results

The spray-dried powder was shown in SEM images to be discreet spheres being observed which were predominantly sub-micron to around 6 or 7 microns in diameter. These images are shown in FIG. 4.

EDX analysis again showed the presence Carbon, Oxygen, Sodium and Nitrogen. The presence of nitrogen is indicative of the nitrite component and the presence of carbon is indicative of the presence of citrate. The EDX analysis is shown in FIG. 5.

The images in FIG. 6 show EDX maps for Nitrogen (green) against a backscattered electron image (BSE), and Nitrogen against Carbon (blue) at a microscope magnification of ×2000 for the spray dried formulation. The associated BSE image is included also to identify particulate features. At this increased magnification, the homogeneity of this spray dried formulation is evident, with individual particulates appearing to show contributions from both Carbon and Nitrogen.

1. A solid powder composition comprising one or more particles containing a nitrite salt and a proton source within the same particle.

2. A solid powder composition comprising particles coated in a hydrophobic material, wherein the coated particles are a particle containing a nitrite salt and a proton source within the same particle and coated with the hydrophobic material.

3. A pharmaceutical composition including the solid powder composition of claim 1 or 2, and optionally one or more excipients and/or adjuvants.

4. A method of preparing a solid powder composition comprising particles coated in a hydrophobic material, the method includes the step of:
(i) coating particles containing a nitrite salt and a proton source within the same particle with a hydrophobic material.

5. A solid powder composition according to claim 1 or 2 or a pharmaceutical composition according to claim 3 for use in a method of treating or preventing a respiratory disease or disorder.

6. A material comprising a substrate and the solid powder composition according to claim 1 or 2, wherein particles of the solid powder composition are incorporated or encapsulated into the substrate.

7. A method of incorporating or encapsulating the solid powder composition according to claim 1 or 2 into a substrate, the method includes the steps of (i) mixing the solid powder composition according to claim 1 or 2 with a non-aqueous liquid containing the substrate or substrate precursor to form a liquid-particle mixture and (ii) solidifying the liquid-particle mixture to form a material incorporating or encapsulating the solid powder composition of claim 1 or 2.
